(19) United States
(12) Patent Application Publication
(10) Pub. No.: US 2019/0015071 A1
(43) Pub. Date: Jan. 17, 2019

(54) ULTRASONIC PROBE

(71) Applicant: Konica Minolta Inc., Chiyoda-ku Tokyo (JP)

(72) Inventors: KIYOSHI FUJII, Toyonaka-shi Osaka (JP); TOSHIHARU SATO, Machida-shi Tokyo (JP); KOJI OURA, Midori-ku Yokohama-shi Kanagawa (JP)

(21) Appl. No.: 16/032,621

(22) Filed: Jul. 11, 2018

(30) Foreign Application Priority Data
Jul. 14, 2017 (JP) 2017-137826

(51) Int. Cl. A61B 8/00; A61B 8/12; G10K 11/02; G01N 29/24

(52) U.S. Cl. CPC A61B 8/4444; G01N 29/2437; G10K 11/02; A61B 8/12

(57) ABSTRACT

An ultrasonic probe includes: a piezoelectric element that transmits and receives an ultrasonic wave; a housing that accommodates the piezoelectric element; and an acoustic medium liquid that contains an aryl group-containing siloxane compound, has an attenuation factor of ultrasonic waves of 5 MHz of less than 1.5 dB/cm, and fills a space between the piezoelectric element and the housing.

### CROSS-REFERENCE TO RELATED APPLICATION

The present application claims priority under 35 U.S.C. § 119 to Japanese patent Application No. 2017-137826, filed on Jul. 14, 2017, the entire contents of which are incorporated herein by reference.

### BACKGROUND

### Technological Field

The present invention relates to an ultrasonic probe used for ultrasonic diagnosis.

### Description of the Related Art

With use of an ultrasonic diagnostic device, a shape and motion of tissues can be obtained as an ultrasonic diagnostic image by a simple operation of applying an ultrasonic probe that is connected to the ultrasonic diagnostic device or constituted so as to be capable of communicating with the ultrasonic diagnostic device onto the body surface or a simple operation of inserting the ultrasonic probe into the body. The ultrasonic diagnostic device is highly safe, therefore has an advantage that inspection can be repeatedly performed.

The ultrasonic probe includes a tip storage part in which a piezoelectric element that transmits and receives ultrasonic waves, and the like are installed, and a grip part for grasping and operating the entire ultrasonic probe. The piezoelectric element receives an electric signal (transmission signal) from an ultrasonic diagnostic device, converts the received transmission signal into an ultrasonic wave signal, transmits the ultrasonic wave signal, receives the ultrasonic wave reflected in a living body, converts the ultrasonic wave into an electric signal (reception signal), and transmits the reception signal converted into the electric signal to the ultrasonic diagnostic device.

Among ultrasonic probes, one that mechanically rotates or swings a piezoelectric element is known because of enabling the scanning in a wide range of a subject (such an ultrasonic probe is hereinafter also referred to as a “mechanical scanning-type ultrasonic probe”). In the mechanical scanning-type ultrasonic probe, a piezoelectric element, and a swing mechanism part for rotating or swinging the piezoelectric element are arranged in a tip storage part. In the tip storage part, a window including a material through which ultrasonic waves are easily transmitted is arranged on a face opposite to a transmission and reception wavefront of the piezoelectric element, and an acoustic medium liquid is filled in a gap between the transmission and reception wavefront of the piezoelectric element and the window.

The acoustic medium liquid is for acoustically matching the transmission and reception wavefront of a piezoelectric element and the window, and transmits and receives ultrasonic waves effectively. In principle, it is sufficient that the acoustic medium liquid is filled only in a gap between the transmission and reception wavefront of a piezoelectric element and the window. However, it is practically difficult to fill only the gap with the acoustic medium liquid, and the space in which a piezoelectric element is installed is liquid-tightly closed, and the liquid-tightly closed space is filled with the acoustic medium liquid in many cases.

As the acoustic medium liquid, a hydrocarbon-based oil is widely used in the conventional technique. For example, in JP 2001-299748 A, in order to improve attenuation of ultrasonic wave signals in an acoustic medium liquid having high viscosity, a hydrocarbon-based oil having a kinematic viscosity of 20 mm²/s or less is used. Further, in JP 2013-198645 A, in order to easily move a friction moving member due to the frictional resistance of an acoustic medium liquid, a hydrocarbon-based oil having a viscosity of 10 to 20 mPa·s is used.

On the other hand, in JP 60-164245 A, as an acoustic medium liquid having an impedance such that sound velocity of ultrasonic waves is the same as that in the living body, a high phenyl silicone oil having five phenyl groups is used.

As a material for the window, polymethylpentene having an acoustic impedance that is close to that of the living body may be used in some cases. However, in JP 1-242041 A, polymethylpentene mixed with a silicone-based oil is used in order to obtain the sound velocity close to sound velocity of the living body. In JP 2001-178727 A, polymethylpentene having mechanical strength that has been improved by mixing a resin modifier to the polymethylpentene is used.

However, when the acoustic medium liquid described in each of JP 2001-299748 A, JP 2013-198645 A, and JP 60-164245 A is used for a conventional window material such as polymethylpentene, ultrasonic waves transmitted from a piezoelectric element (first transmission) have sometimes reflected between the acoustic medium liquid and the window. The reflected ultrasonic waves are further reflected by a piezoelectric element or the like, and are transmitted (second transmission) toward the inside of the living body. Therefore, the piezoelectric element receives multiple ultrasonic waves that have propagated multiply and have respectively reflected in the living body. As a result, noise (artifact) is superimposed on the ultrasonic image to be obtained, and the accuracy is deteriorated.

### SUMMARY

In view of the above-described problems, an object of the present invention is to provide an ultrasonic probe capable of suppressing the occurrence of noise (artifact) due to multiple reflections, and an ultrasonic diagnostic device equipped with the ultrasonic probe.

To achieve the abovementioned object, according to an aspect of the present invention, an ultrasonic probe reflecting one aspect of the present invention comprises: a piezoelectric element that transmits and receives an ultrasonic wave; a housing that accommodates the piezoelectric element; and an acoustic medium liquid that contains an aryl group-containing siloxane compound, has an attenuation factor of ultrasonic waves of 5 MHz of less than 1.5 dB/cm, and fills a space between the piezoelectric element and the housing.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is an external perspective view of an ultrasonic diagnostic device using an ultrasonic probe;

FIG. 2 is a sectional view showing the overall structure of an ultrasonic probe;

FIG. 3 is an enlarged sectional view of a tip storage part;

FIG. 4A is a graph showing the relationship between the immersion time and the mass change ratio of a test sample formed of silicone rubber when the test sample formed of silicone rubber is immersed in a hydrocarbon-based oil;

FIG. 4B is a graph showing the relationship between the immersion time and the mass change ratio of a test sample formed of silicone rubber when the test sample formed of silicone rubber is immersed in benzyltoluene;

FIG. 4C is a graph showing the relationship between the immersion time and the mass change ratio of a test sample formed of silicone rubber when the test sample formed of silicone rubber is immersed in a methyl phenyl silicone oil;

FIG. 4D is a graph showing the relationship between the immersion time and the mass change ratio of a test sample formed of polymethylpentene when the test sample formed of polymethylpentene is immersed in a hydrocarbon-based oil;

FIG. 4E is a graph showing the relationship between the immersion time and the mass change ratio of a test sample formed of polymethylpentene when the test sample formed of polymethylpentene is immersed in benzyltoluene; and

FIG. 4F is a graph showing the relationship between the immersion time and the mass change ratio of a test sample formed of polymethylpentene when the test sample formed of polymethylpentene is immersed in a methyl phenyl silicone oil.

### DETAILED DESCRIPTION OF EMBODIMENTS

Hereinafter, one or more embodiments of the present invention will be described with reference to the drawings. However, the scope of the invention is not limited to the disclosed embodiments.

(Ultrasonic Diagnostic Device)

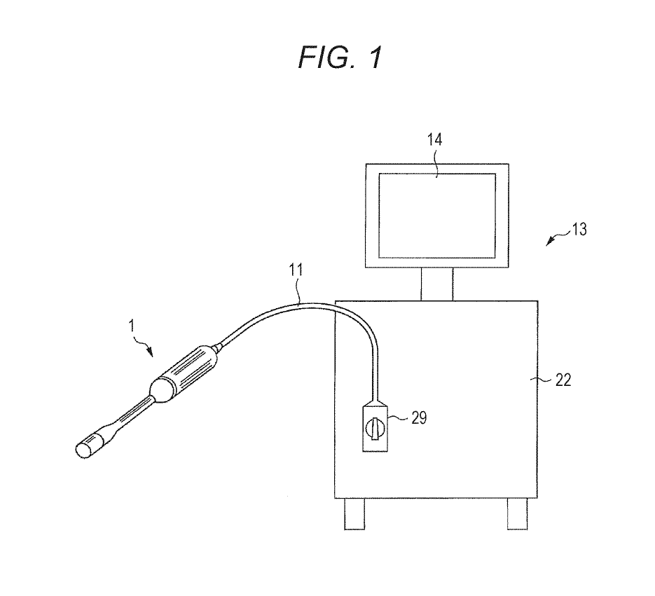

FIG. 1 is an external perspective view of an ultrasonic diagnostic device 13 equipped with the ultrasonic probe 1 according to the present embodiment.

The ultrasonic diagnostic device 13 is equipped with a main body part 22, a connector part 29, and a display 14.

The ultrasonic probe 1 is connected to the ultrasonic diagnostic device 13 via a cable 11 connected to the connector part 29.

An electric signal (transmission signal) from the ultrasonic diagnostic device 13 is transmitted to a piezoelectric element (described later) of the ultrasonic probe 1 through the cable 11. This transmission signal is converted into an ultrasonic wave in the piezoelectric element, and is transmitted into the living body. The transmitted ultrasonic wave is reflected from the tissues or the like in the living body, and a part of the reflected waves is received by the piezoelectric element, converted into an electric signal (reception signal), and transmitted to the ultrasonic diagnostic device 13. The received signal is converted into image data by the ultrasonic diagnostic device 13, and is displayed on the display 14.

(Ultrasonic Probe)

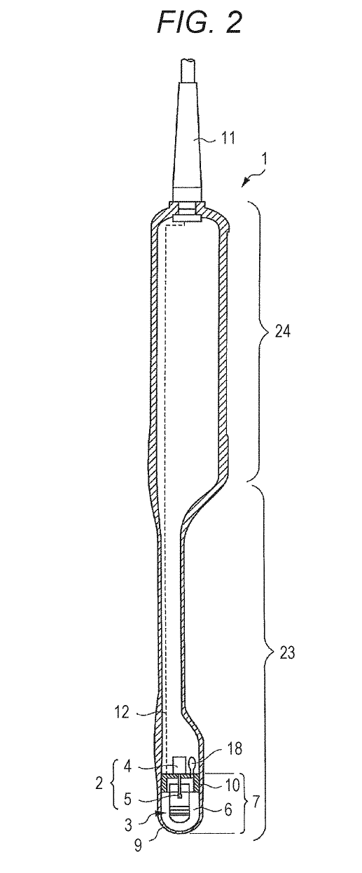

FIG. 2 is a sectional view showing one example of the overall structure of an ultrasonic probe 1. The ultrasonic probe 1 is a probe used for ultrasonic diagnosis, and is a body cavity insertion-type probe capable of performing scanning in the body cavity with ultrasonic waves by inserting a part of the probe into the body cavity of a subject.

As shown in FIG. 2, the ultrasonic probe 1 is equipped with an insertion part 23 including a tip storage part 7, which is inserted into the body cavity, and a grip part 24 that is gripped by an operator in the outside of the body cavity, and is constituted to be connectable to a cable 11 that is connected to a main body 22. Multiple signal lines 12 are drawn out from the tip storage part 7, and are connectable to the cable 11 through the insertion part 23 and the grip part 24.

Such a body cavity insertion-type probe is frequently used by being inserted into the body cavity of a subject, but in general there is also an ultrasonic probe that is used by being applied onto the body surface without being inserted into the body cavity of a subject. Note that the ultrasonic probe according to the present invention is not limited to a body cavity insertion-type probe.

In addition, the ultrasonic probe 1 is constituted to be connectable to the ultrasonic diagnostic device 13 via the cable 11, but may be constituted to be connectable to the ultrasonic diagnostic device 13 by wireless communication without arranging a cable.

Next, the tip storage part 7 will be described in detail.

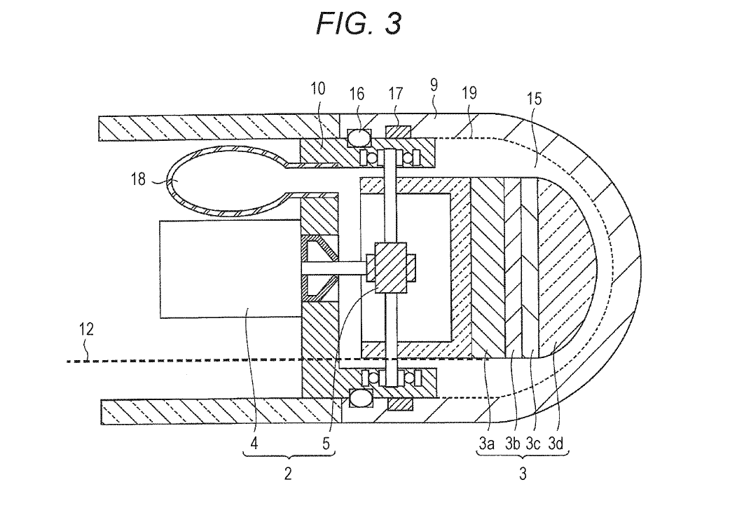

FIG. 3 is an enlarged sectional view of the tip storage part 7 shown in FIG. 2. The tip storage part 7 is constituted by joining a window 9 that forms a part of a housing of the ultrasonic probe 1 to a frame 10 that is a holding member, and is equipped with a piezoelectric element unit 3, a swing mechanism part 2 for holding and swinging the piezoelectric element unit 3, and an internal space 15 that is filled with an acoustic medium liquid 6 for transmitting an ultrasonic wave signal.

The window 9 is a protective member for protecting the piezoelectric element unit 3 and the like from a pressure due to the contact with the living body, and is arranged in a position covering the tip storage part 7 on the side in contact with the living body.

The frame 10 is sealed so as to be in close contact with the inner wall of the window 9 by a sealing member 16 such as an O-ring or a packing, an adhesive 17, and the like, and with this arrangement, the inner part of the tip storage part 7 is liquid-tightly sealed. As the frame 10, for example, one including metal or resin can be used. In a case of metal, for example, one including aluminum can be used. In a case of resin, it is desired to use a resin that does not swell due to the contact with an acoustic medium liquid 6 described later. Further, in the frame 10, a wiring hole (not shown) through which the multiple signal lines 12 described above are passed is arranged. In order to maintain the sealed state of the tip storage part 7, in the wiring hole, the signal lines 12 and the frame 10 are liquid-tightly sealed with an adhesive or the like.

As shown in FIG. 3, the piezoelectric element unit 3 is constituted by laminating a backing layer 3a, a piezoelectric element 3b, an acoustic matching layer 3c, and an acoustic lens 3d.

The backing layer 3a is arranged on a surface of the piezoelectric element 3b on the side opposite to the living body side, supports the piezoelectric element 3b, and absorbs the ultrasonic waves transmitted to the side opposite to the living body side of the piezoelectric element 3b. As a material for the backing layer 3a, for example, natural rubber, epoxy resin, thermoplastic resin, or the like can be used.

The piezoelectric element 3b is a layer constituted of a piezoelectric material. Examples of the piezoelectric material include lead zirconate titanate (PZT), piezoelectric ceramic, lead zincate niobate titanate (PZNT), and lead magnesium niobate titanate (PMNT). The thickness of the piezoelectric element 3b can be set to, for example, 0.05 mm or more to 0.4 mm or less. On a surface on the living body side of the piezoelectric element 3b and on a surface on the side opposite to the living body side, electrodes (not shown) for applying a voltage to the piezoelectric element 3b are arranged. These electrodes are connected to signal lines 12, and transmit electric signals to and receive electric signals from the piezoelectric element 3b.

The acoustic matching layer 3c is a layer for matching the acoustic characteristics of the piezoelectric element 3b and the acoustic lens 3d, and is constituted of a material having an acoustic impedance mostly intermediate between the piezoelectric element 3b and the acoustic lens 3d. The acoustic matching layer 3c may be a single layer or a lamination layer. However, from the viewpoint of adjusting the acoustic characteristics, it is preferred that the acoustic matching layer 3c is a laminate of multiple layers (for example, two or more layers, and more preferably four or more layers) having different acoustic impedances, and it is more preferred that the layers are arranged toward the acoustic lens 3d so that the acoustic impedance of each of the layers is set to gradually or continuously approach to the acoustic impedance of the acoustic lens 3d. In addition, the layers of the acoustic matching layer 3c may be bonded to each other with an adhesive (for example, an epoxy-based adhesive) that is usually used in the technical field.

The acoustic matching layer 3c can be constituted of various materials. As these materials, for example, aluminum, an aluminum alloy, a magnesium alloy, macole glass, glass, fused quartz, copper graphite, a resin, or the like can be used. Examples of the resin include polyethylene, polypropylene, polycarbonate, an ABS resin, an AAS resin, an AES resin, nylon, polyphenylene oxide, polyphenylene sulfide, polyphenylene ether, polyether ether ketone, polyamideimide, polyethylene terephthalate, an epoxy resin, and a urethane resin.

The acoustic lens 3d is constituted of, for example, a soft polymer material having an acoustic impedance mostly intermediate between the acoustic matching layer 3c and the living body, focuses ultrasonic beams by utilizing the refraction, and improves the resolution. Examples of the soft polymer material include silicone-based rubber, butadiene-based rubber, polyurethane rubber, epichlorohydrin rubber, and ethylene-propylene copolymer rubber obtained by copolymerizing ethylene and propylene. Among them, silicone-based rubber, and butadiene-based rubber are preferred, and from the viewpoint of the characteristics of an acoustic lens, silicone rubber belonging to silicone-based rubber, and butadiene rubber belonging to butadiene-based rubber are particularly preferred.

The swing mechanism part 2 is equipped with a transmission mechanism part 5 for holding and swinging a piezoelectric element unit 3, and a motor 4 for driving the rotation of a gear (transmission mechanism) in the transmission mechanism part 5. In conjunction with the rotation of the gear (transmission mechanism) in the transmission mechanism part 5, the swing mechanism part 2 swings the piezoelectric element unit 3 to scan the ultrasonic wave signals. In addition, a rotation mechanism part (not shown) for holding and rotating a piezoelectric element unit 3 may be arranged together with or in place of the swing mechanism part 2 for holding and swinging the piezoelectric element unit 3. Further, in the transmission mechanism part 5, in addition to the gears, for example, a timing belt, a wire, or the like can be used as a transmission mechanism for swinging the piezoelectric element unit 3.

The internal space 15 is a space liquid-tightly closed by a window 9 and a frame 10, and houses an acoustic medium liquid 6.

The ultrasonic wave transmitted from the piezoelectric element 3b propagates through the respective media in the order of the acoustic matching layer 3c, the acoustic lens 3d, the acoustic medium liquid 6, and the window 9, and reaches the living body. The ultrasonic wave reflected from the tissues in the living body propagates through the respective media in the reverse order, and is received by the piezoelectric element 3b.

The acoustic medium liquid 6 contains an aryl group-containing siloxane compound, and is a liquid having an attenuation factor of ultrasonic waves of 5 MHz of less than 1.5 dB/cm.

The aryl group-containing siloxane compound is sufficient as long as it has a siloxane skeleton and an aryl group. The siloxane skeleton may be linear, branched or cyclic. The aryl group may have an aromatic ring, and may be any of a monocyclic ring, a condensed ring, and a heterocyclic ring. In the aryl group-containing siloxane compound, the intermolecular distance is small due to the π-π bond between aromatic rings and the density can become high, therefore, it is considered that the acoustic impedance can be made larger than that of the hydrocarbon-based oil. The aryl group is preferably a phenyl group hardly causing the steric hindrance that hinders high density.

Examples of the aryl group-containing siloxane compound include compounds mentioned by the following general formulas (1), (2) and (3).

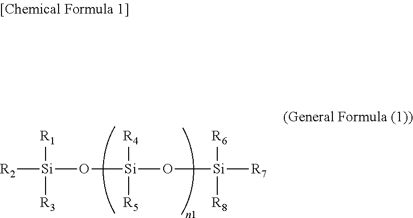

In the general formula (1), R₁ to R₈ independently represent a hydrogen atom, a hydroxyl group, an alkyl group, or an aryl group, and at least one of R₁ to R₈ is an aryl group. The alkyl group is a linear or branched alkyl group having 1 or more to 20 or less carbon atoms, which is substituted or unsubstituted, the aryl group is a phenyl group, a naphthyl group, an anthryl group, or a phenanthryl group, which is substituted or unsubstituted, or an aralkyl group in which these groups are bonded to a linear or branched alkyl group having 1 or more to 20 or less carbon atoms, which is substituted or unsubstituted, and n1 is an integer of 1 or more to 1000 or less. The substituent to be substituted for the alkyl group or the aryl group is a halogen atom, and an alkyl group having 1 or more to 20 or less carbon atoms.

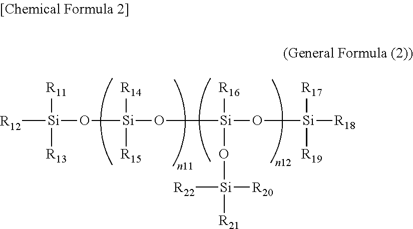

In the general formula (2), R₁₁ to R₂₂ independently represent a hydrogen atom, a hydroxyl group, an alkyl group, or an aryl group, and at least one of R₁₁ to R₂₂ is an aryl group. The alkyl group is a linear or branched alkyl group having 1 or more to 20 or less carbon atoms, which is substituted or unsubstituted, the aryl group is a phenyl group, a naphthyl group, an anthryl group, or a phenanthryl group, which is substituted or unsubstituted, or an aralkyl group in which these groups are bonded to a linear or branched alkyl group having 1 or more to 20 or less carbon atoms, which is substituted or unsubstituted, and n11 and n12 are independently an integer of 1 or more to 1000 or less. The substituent to be substituted for the alkyl group or the aryl group is a halogen atom, and an alkyl group having 1 or more to 20 or less carbon atoms.

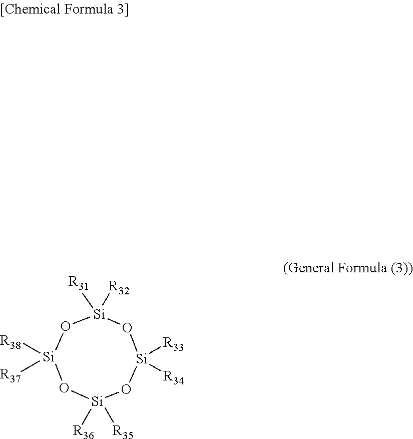

In the general formula (3), R₃₁ to R₃₈ independently represent a hydrogen atom, a hydroxyl group, an alkyl group, or an aryl group, and at least one of R₃₁ to R₃₈ is an aryl group. The alkyl group is a linear or branched alkyl group having 1 or more to 20 or less carbon atoms, which is substituted or unsubstituted, the aryl group is a phenyl group, a naphthyl group, an anthryl group, or a phenanthryl group, which is substituted or unsubstituted, or the alkyl group is an aralkyl group in which these groups are bonded to a linear or branched alkyl group having 1 or more to 20 or less carbon atoms, which is substituted or unsubstituted. The substituent to be substituted for the alkyl group or the aryl group is a halogen atom, and an alkyl group having 1 or more to 20 or less carbon atoms.

The substituents R₁ to R₈, R₁₁ to R₂₂, and R₃₁ to R₃₈ in the general formulas (1) to (3) each preferably have at least one phenyl group, more preferably have 2 or more to 5 or less of phenyl groups, and furthermore preferably have four phenyl groups. Further, among the substituents R₁ to R₈, R₁₁ to R₂₂, and R₃₁ to R₃₈, the substituent other than the phenyl group is preferably an alkyl group, and more preferably a methyl group. Hereinafter, a compound in which all of the substituents R₁ to R₈, R₁₁ to R₂₂, or R₃₁ to R₃₈ are any of phenyl groups or methyl groups is referred to as “methylphenyl silicone”.

The numbers of repeating units, n1, n11 and n22 in the general formulas (1) and (2) each are preferably 1 or more to 10 or less, more preferably 1 or more to 5 or less, furthermore preferably 1 or more to 3 or less, and particularly preferably 1.

In Table 1, properties of typical aryl group-containing siloxane compounds, a hydrocarbon-based oil, and benzyltoluene are shown. Note that the properties of the hydrocarbon-based oil are the values described in JP 2001-299748 A.

The values of the density, the sound velocity, the acoustic impedance, the kinematic viscosity at 40° C., and the attenuation factor of ultrasonic waves are values obtained by the measurement using the following method. Note that each value of the above properties in the present specification is a value obtained by the measurement using the following method, unless otherwise noted.

The value of the density is a value obtained by the measurement using an electronic densimeter SD-200L (manufactured by Alfa Mirage Co., Ltd.) in accordance with a density measurement method of Method A (displacement of water method) described in JIS-K7112 02.

The value of the sound velocity is a value obtained by the measurement at 25° C. using a sing-around type sound velocity measurement device manufactured by ULTRASONIC ENGINEERING CO., LTD. in accordance with JIS Z2353-2003.

The value of the acoustic impedance is a value obtained by the density and the sound velocity in accordance with the following equation.

Acoustic impedance (Mrayl)=density (×10³ kg/m³)×sound velocity (×10³ m/sec)

The value of the kinematic viscosity is a value obtained by the measurement at 40° C. using Mini-AV-X manufactured by STM Corporation, which is a measurement instrument in accordance with JIS K2283.

The value of the attenuation factor of ultrasonic waves is a value obtained in accordance with JIS Z2354-1992 by generating ultrasonic waves of 5 MHz using an ultrasonic pulser & receiver JPR-10C (manufactured by JAPAN PROBE CO., LTD.) in a water tank filled with water at 25° C., and then by measuring the amplitudes of the ultrasonic waves before and after the ultrasonic waves transmit through a sheet.

TABLE 1

| | Acoustic medium liquid | | | | | |
|---|---|---|---|---|---|---|
| | Aryl group-containing siloxane compound (methylphenyl silicone) | | | | | |
| | General | | General | General | Hydrocarbon-based | |
| | Formula (1) | General | Formula (1) | Formula (1) | oil (see JP | |
| | phenyl group × 2 | Formula (3) | phenyl group × 4 | phenyl group × 5 | 2001-299748 A) | Benzyltoluene |
| Structure | n1 = 1 | phenyl group × 2 | n1 = 1 | n1 = 1 | — | — |
| Density (kg/m³) | 1.02 | 1.104 | 1.065 | 1.107 | 0.85 | 1.02 |
| Sound velocity (m/s) | 1238 | 1294 | 1408 | 1484 | 1400 | 1497 |
| Acoustic impedance | 1.26 | 1.43 | 1.50 | 1.64 | 1.19 | 1.5 |
| (Mrayl) | | | | | | |
| Kinematic viscosity at | 11 | 23 | 21 | 90 | 15 | 2.5 |
| 40° C. (mm²/s) | | | | | | |
| Attenuation factor of | 1.0 | 0.90 | 0.85 | 4.0 | 1.19 | 0.13 |
| ultrasonic wave (5 MHz) | | | | | | |
| (dB/cm) | | | | | | |

When propagating between different media, the ultrasonic waves are reflected in proportion to the difference in acoustic impedance between the media. In the present example, since a material having an acoustic impedance that is close to that of the living body is used for the window 9 as described above, when the acoustic impedance of the acoustic medium liquid 6 is also closer to that of the living body, reflection of ultrasonic waves between the acoustic medium liquid 6 and the window 9 is suppressed, noise (artifact) caused by multiple propagation of ultrasonic waves in the living body due to the reflection is suppressed, and an ultrasonic image with the improved accuracy is obtained.

The hydrocarbon-based oil as described in JP 2001-299748 A, and JP 2013-198645 A has a low viscosity, therefore, the acoustic impedance that is obtained by the product of the density of a medium and the sound velocity also becomes low. In general, the hydrocarbon-based oil has a sound velocity of around 1400 to 1450 m/s, therefore, the acoustic impedance is generally 1.2 MRayl, and the difference from the acoustic impedance (around 1.53 MRayl) of the living body is large.

On the other hand, the aryl group-containing siloxane compound used as an acoustic medium liquid 6 in the present embodiment has a density higher than that of the hydrocarbon-based oil, and has an acoustic impedance closer to that of the living body.

In addition, it is considered that if a hydrocarbon-based oil having a larger average molecular weight is used, the acoustic impedance of the acoustic medium liquid 6 can also be made larger. However, when a hydrocarbon-based oil having a larger average molecular weight is used, the kinematic viscosity of the acoustic medium liquid 6 increases, the physical load on the piezoelectric element unit 3 during the swinging increases, and further the scanning at high speed may become difficult.

On the other hand, the aryl group-containing siloxane compound used as an acoustic medium liquid 6 in the present embodiment has an acoustic impedance closer to that of the living body, and further has a low kinematic viscosity at 40° C. Therefore, when an aryl group-containing siloxane compound is used alone, or used as a mixture in appropriate combination, the viscosity at 40° C. is 30 mm²/s or less, and preferably 22 mm²/s or less, so that the mechanical load on the piezoelectric element unit 3 is reduced, and further the scanning at a high speed is easily performed.

When the attenuation factor of ultrasonic waves is large, the acoustic medium liquid 6 may lower the test depth of the ultrasonic diagnosis or may lower the brightness of the image, and as a result, the accuracy of the ultrasonic diagnostic image may be deteriorated.

The hydrocarbon-based oil as described in JP 2001-299748 A, and JP 2013-198645 A has an attenuation factor of ultrasonic waves of around 1.19 dB/cm, and is to the extent capable of withstanding the practical use, however, when the acoustic impedance is increased, the attenuation factor of ultrasonic waves also tends to be increased. Therefore, it is difficult to achieve a balance between the acoustic impedance and the attenuation factor of ultrasonic waves.

On the other hand, the aryl group-containing siloxane compound used as an acoustic medium liquid 6 in the present embodiment has an acoustic impedance closer to that of the living body, and further has a small attenuation factor of ultrasonic waves. Accordingly, when an aryl group-containing siloxane compound is used alone, or used as a mixture in appropriate combination, it is easy to suppress the deterioration of the accuracy of the ultrasonic diagnostic image by setting the attenuation factor of ultrasonic waves of 5 MHz of the acoustic medium liquid 6 to be less than 1.5 dB/cm.

When the boiling point of the acoustic medium liquid 6 is low, the acoustic medium liquid 6 easily volatilizes, and air bubbles tend to occur inside the sealed internal space 15. The air bubbles and the like mixed in the acoustic medium liquid 6 cause the hindrance of propagation of ultrasonic waves. Therefore, as the acoustic medium liquid 6, a liquid in which the phase change from a liquid to a gas hardly occurs and the properties are stable over time is required.

Water is excellent in the acoustic characteristic when used as the acoustic medium liquid 6 because of having an acoustic impedance of around 1.45 Mrayl, and a small attenuation factor of ultrasonic waves. However, water has a low boiling point, and easily volatilizes, therefore, air bubbles easily occur.

On the other hand, the aryl group-containing siloxane compound used as an acoustic medium liquid 6 in the present embodiment has high boiling point, and further is structurally stable, therefore, air bubbles hardly occur.

The acoustic medium liquid 6 is in contact with a window 9, an acoustic lens 3d of a piezoelectric element unit 3, an adhesive and the like inside the internal space 15, therefore, is required that the chemical attack on the materials constituting these members is small.

With regard to this, the aryl group-containing siloxane compound used as an acoustic medium liquid 6 in the present embodiment has an extremely smaller chemical attack on silicone rubber and poly-α-olefin that are used as a material for a window 9, silicone rubber and polystyrene that are used as a material for an acoustic lens 3d, nitrile rubber, silicone rubber, chloroprene rubber, and fluorine rubber that are used as a material for a reservoir 18, an epoxy-based adhesive that is used as an adhesive, and the like even as compared with that of a hydrocarbon-based oil, benzyltoluene, or the like.

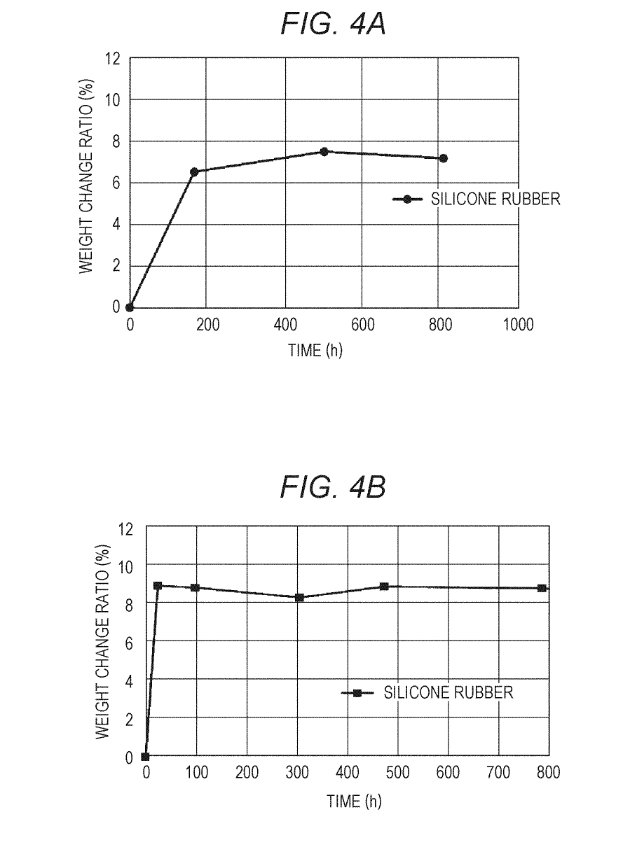

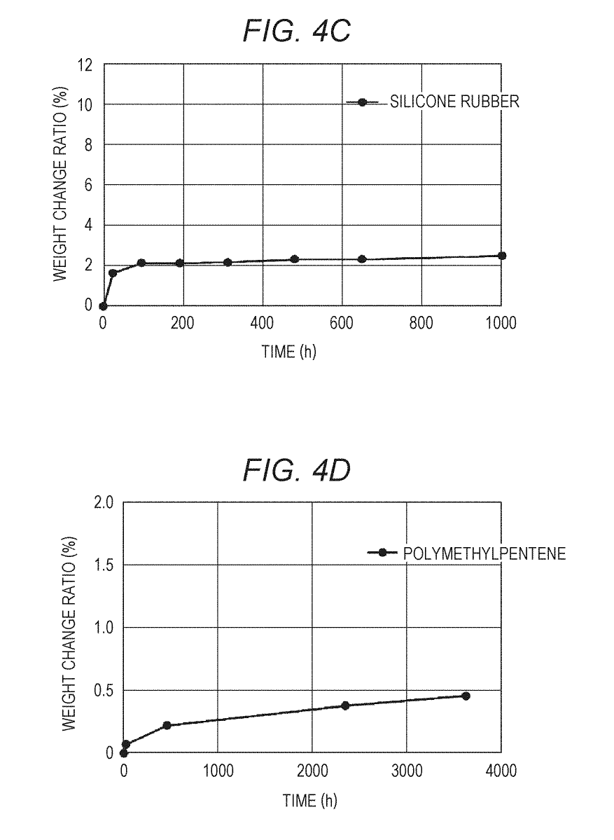

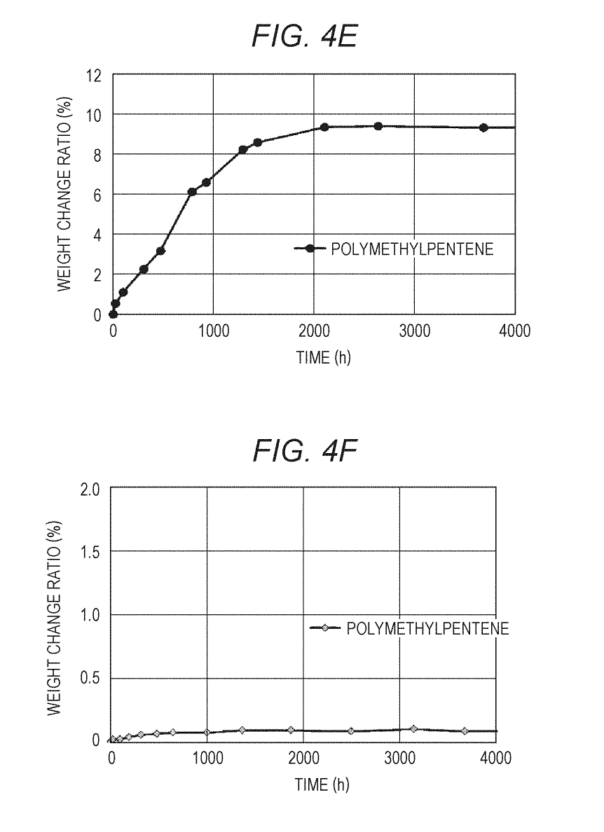

FIG. 4A to FIG. 4F are graphs showing test results of the chemical attack on a material of window 9 with various kinds of acoustic medium liquids.

The test was performed by the following procedure. Silicone rubber and polymethylpentene were prepared. A sample of the silicone rubber in an amount of 5 g was cured at room temperature, then the cured sample was further left to stand at room temperature for 48 hours to be completely cured, and the completely cured sample was taken as a test sample. As for the polymethylpentene, a piece of 5 g was cut out from the test piece obtained by injection molding, and the piece of 5 g was taken as a test sample. The initial mass of each of the completely cured test samples was measured with an electronic balance, and then each of the test samples was immersed in various kinds of medium liquids. As the medium liquid, a hydrocarbon-based oil, benzyltoluene, or methylphenyl silicone was used. After that, every time the predetermined period of time elapses, a test sample was taken out from the medium liquid, and the mass of the test sample at that time was measured with an electronic balance. FIG. 4A to FIG. 4F are graphs for each of the test samples, obtained by plotting the mass reduction rate of each of the test samples by every lapse of time with the immersion time on the horizontal axis, and with the mass change ratio (the value (%) obtained by dividing the mass reduction amount at the time of measurement from the initial mass by the initial mass) on the vertical axis.

FIG. 4A is a graph showing the relationship between the immersion time and the mass change ratio of a test sample formed of silicone rubber when the test sample is immersed in hydrocarbon-based oil, FIG. 4B is a graph showing the relationship between the immersion time and the mass change ratio of a test sample formed of silicone rubber when the test sample is immersed in benzyltoluene, and FIG. 4C is a graph showing the relationship between the immersion time and the mass change ratio of a test sample formed of silicone rubber when the test sample is immersed in methyl phenyl silicone oil.

FIG. 4D is a graph showing the relationship between the immersion time and the mass change ratio of a test sample formed of polymethylpentene when the test sample is immersed in hydrocarbon-based oil, FIG. 4E is a graph showing the relationship between the immersion time and the mass change ratio of a test sample formed of polymethylpentene when the test sample is immersed in benzyltoluene, and FIG. 4F is a graph showing the relationship between the immersion time and the mass change ratio of a test sample formed of polymethylpentene when the test sample is immersed in methyl phenyl silicone oil.

As shown in FIG. 4A, FIG. 4B, and FIG. 4C, when a test sample formed of silicone rubber was immersed in methylphenyl silicone, the mass reduction rate of the test sample was smaller than that when a test sample was immersed in each of hydrocarbon-based oil and benzyltoluene.

As shown in FIG. 4D, FIG. 4E, and FIG. 4F, when a test sample formed of polymethylpentene was immersed in methylphenyl silicone, the mass reduction rate of the test sample was smaller than that when a test sample was immersed in each of hydrocarbon-based oil and benzyltoluene.

From these results, it can be understood that the methylphenyl silicone has a small chemical attack on both of the silicone rubber and polymethylpentene.

As the acoustic medium liquid 6, the above-described aryl group-containing siloxane compound may be used singly alone, however, in order to adjust various properties of the acoustic medium liquid 6 to a desired extent, an aryl group-containing siloxane compound and another medium liquid, or multiple kinds of aryl group-containing siloxane compounds may be used in combination.

The acoustic medium liquid 6 obtained by mixing an aryl group-containing siloxane compound and a hydrocarbon-based oil can further reduce the kinematic viscosity and the attenuation of ultrasonic waves by the hydrocarbon-based oil having a low viscosity and further less attenuation of ultrasonic waves in addition to the reduction of multiple reflections by the aryl group-containing siloxane compound having an acoustic impedance that is close to the living body. In order to obtain the similar effect, an acoustic medium liquid 6 obtained by mixing an aryl group-containing siloxane compound and a silicone oil may be used, or an acoustic medium liquid 6 obtained by mixing an aryl group-containing siloxane compound, a hydrocarbon-based oil, and a silicone oil may be used.

From the viewpoint of making the acoustic impedance closer to that of the living body, the acoustic medium liquid 6 preferably contains an aryl group-containing siloxane compound having 2 or more to 5 or less of phenyl groups, and more preferably contains an aryl group-containing siloxane compound having 4 phenyl groups. In addition, in order to adjust the various properties including the acoustic impedance, the attenuation factor of ultrasonic waves, and the like, the acoustic medium liquid 6 preferably contains multiple aryl group-containing siloxane compounds each having a different number of aromatic rings. The aryl group-containing siloxane compound contained in the various kinds of acoustic medium liquids 6 is preferably methylphenyl silicone.

As the material for a window 9, a material that is hardly deformed by pressing (contact with the living body), such as silicone rubber, or poly-α-olefin can be used. The material has an acoustic impedance larger than that of the living body in many cases, therefore, a poly-α-olefin having a smaller density and a smaller acoustic impedance is preferably used, and polymethylpentene that has an acoustic impedance of 1.67 Mrayl and is closer to that of the living body is more preferably used. By using a material, which has an acoustic impedance close to that of the living body, for the window 9, multiple reflections between the acoustic medium liquid 6 and the window 9 and between the window 9 and the living body can be suppressed.

Further, as in the comparison between FIG. 4C and FIG. 4F, as the material for a window 9, a poly-α-olefin is preferred, and polymethylpentene is more preferred also from the viewpoint of reducing the chemical attack by the aryl group-containing siloxane compound that is contained in an acoustic medium liquid 6.

The poly-α-olefin, in particular polymethylpentene can make the acoustic impedance closer to that of the living body by mixing a plasticizer. As the plasticizer, from the viewpoint of suppressing the swelling of the window 9 due to the acoustic medium liquid 6, the precipitation of the plasticizer on a surface of the window 9, and the permeation of the acoustic medium liquid 6 through the window 9, a hydrocarbon-based oil that has low compatibility with an aryl group-containing siloxane compound is preferred. Moreover, as the plasticizer, a poly-α-olefin oil that has high safety to the living body is more preferred because of being saturated and thus chemically stabilized, and further, having less impurities.

The acoustic impedance of the poly-α-olefin, in particular polymethylpentene can be adjusted by the amount of the plasticizer. In Table 2, the relationship between the amount of a plasticizer and the acoustic impedance of a material after kneading is shown when a poly-α-olefin oil that has a weight average molecular weight of 3100 as a plasticizer is kneaded with polymethylpentene.

TABLE 2

| Amount of plasticizer | Acoustic impedance |
|---|---|
| (% by mass) | (Mrayl) |
| 0 | 1.67 |
| 2 | 1.67 |
| 4 | 1.66 |
| 6 | 1.64 |
| 8 | 1.63 |
| 10 | 1.62 |
| 11 | 1.59 |
| 12 | 1.58 |
| 13 | 1.58 |
| 15 | 1.57 |
| 19 | 1.53 |

From Table 2, from the viewpoint of making the acoustic impedance closer to that of the living body, the amount of the plasticizer is preferably 6% by mass or more to 19% by mass or less with respect to the total mass of the material of the window 9. In addition, when the amount of the plasticizer is in the above-described range, the rigidity of a material of the window 9 is moderately lowered, cracking or the like hardly occurs in the window 9, and further the material of the window 9 is not excessively softened.

In addition, as described above, the acoustic medium liquid 6 is filled in an internal space 15 that is liquid-tightly closed, but generally expands and contracts depending on the environmental temperatures. Due to the expansion of the acoustic medium liquid 6, the internal pressure of the internal space 15 rises, and failures such as cracks and liquid leakage may occur.

Further, also in a step of sealing the acoustic medium liquid 6 in the internal space 15, air bubbles may be mixed. When the air bubbles are present between the piezoelectric element unit 3 and the window 9, the hindrance of propagation of ultrasonic waves is caused, and there may be a problem that a clear ultrasonic tomographic image cannot be obtained in some cases due to the attenuation of ultrasonic signals by air bubbles, or due to the occurrence of reflection.

As shown in FIG. 3, in order to prevent such a failure, a reservoir 18 for absorbing expansion and contraction of the acoustic medium liquid 6, which is connected to an internal space 15, may be arranged outside the internal space 15.

As the material for the reservoir 18, a fluorine-based rubber is preferably used because a material such as rubber and resin is easily swelled under the environment of an aryl group-containing siloxane compound.

In addition, since the air bubbles and the acoustic medium liquid 6 have different surface tensions and different specific gravities, a bubble reservoir part (not shown) for moving the air bubbles out of the internal space 15 may be arranged together with or in place of the above-described reservoir 18.

According to the present invention, an ultrasonic image that has less noise (artifact) than that in the conventional ones can be obtained. Therefore, the present invention is expected to expand the range to which ultrasonic diagnosis can be applied.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasonic probe comprising:
a piezoelectric element that transmits and receives an ultrasonic wave;
a housing that accommodates the piezoelectric element; and
an acoustic medium liquid that contains an aryl group-containing siloxane compound, has an attenuation factor of ultrasonic waves of 5 MHz of less than 1.5 dB/cm, and fills a space between the piezoelectric element and the housing.

2. The ultrasonic probe according to claim 1, wherein
the acoustic medium liquid contains an aryl group-containing siloxane compound and a hydrocarbon-based oil.

3. The ultrasonic probe according to claim 1, wherein
the acoustic medium liquid contains an aryl group-containing siloxane compound that has 2 or more to 5 or less of phenyl groups.

4. The ultrasonic probe according to claim 1, wherein
the acoustic medium liquid contains an aryl group-containing siloxane compound that has 4 phenyl groups.

5. The ultrasonic probe according to claim 1, wherein
the acoustic medium liquid contains a plurality of aryl group-containing siloxane compounds each of which has a different number of phenyl groups.

6. The ultrasonic probe according to claim 1, wherein
the acoustic medium liquid fills an internal space that is liquid-tightly sealed by a window forming a part of the housing and a frame of the housing, and
the window includes a material containing a poly-α-olefin.

7. The ultrasonic probe according to claim 6, wherein
the window includes a material that contains polymethylpentene.

8. The ultrasonic probe according to claim 6, wherein
the window includes a material that contains polymethylpentene mixed with a plasticizer.

9. The ultrasonic probe according to claim 8, wherein
the window includes a material that contains polymethylpentene mixed with a poly-α-olefin oil.

10. The ultrasonic probe according to claim 9, wherein
the window includes the material that has a content of the poly-α-olefin oil of 6% or more to 19% or less relative to the total mass.

11. An ultrasonic diagnostic device comprising
the ultrasonic probe according to claim 1.
